(19) United States
(12) Patent Application Publication
(10) Pub. No.: US 2023/0357808 A1
(43) Pub. Date: Nov. 9, 2023

(54) MICROORGANISM AND METHOD FOR THE IMPROVED PRODUCTION OF ALANINE

(71) Applicant: METABOLIC EXPLORER, Saint Beauzire (FR)

(72) Inventors: Laurence DUMON-SEIGNOVERT, Pont Du Chateau (FR); Céline RAYNAUD, Saint Beauzire (FR)

(73) Assignee: METABOLIC EXPLORER, Saint Beauzire (FR)

(21) Appl. No.: 18/023,960

(22) PCT Filed: Sep. 1, 2021
(86) PCT No.: PCT/EP2021/074118

§ 371 Date: Feb. 28, 2023

(30) Foreign Application Priority Data
Sep. 1, 2020 (EP) 20305967.0

(51) Int. Cl. C12N 9/06; C12P 13/06

(52) U.S. Cl. CPC C12P 13/06; C12N 9/0016; C12Y 104/01001

(57) ABSTRACT

The present invention relates to a microorganism genetically modified for improved production of alanine, wherein the microorganism expresses a heterologous alaD gene coding an alanine dehydrogenase and has reduced Lrp transcription factor activity and/or expression. The present invention also relates to a method for the production of alanine using said microorganism.

### FIELD OF INVENTION

The present invention relates to a microorganism genetically modified for the improved production of alanine and to a method for the improved production of alanine using said microorganism.

### BACKGROUND OF THE INVENTION

L-alanine is a non-essential amino acid with numerous industrial applications, in particular in the food industry as a flavor enhancer and/or nutritional supplement as well as in the pharmaceutical industry (e.g. in the context of nutrition therapy and in the synthesis of vitamin B6). It is an odorless, sweet, white solid crystal that is soluble in water and ethanol but not in ether. The global alanine market in 2020 was valued at more than 290 million USD, with production estimated at 500 tons per year (The Expresswire, 2020; Wendisch, 2014).

L-alanine may be manufactured via chemical synthesis (e.g. via Strecker synthesis or the Bucherer-Bergs method variant), enzymatic conversion, or fermentation. That said, chemical methods are of little industrial interest as both D- and L- enantiomers are generated in equimolar amounts, while enzymatic and fermentation methods produce only one enantiomer. L-alanine is generally produced on an industrial scale via the enzymatic decarboxylation of L-aspartic acid using L-aspartate β-decarboxylase from Pseudomonas dacunhae as such or using P. dacunhae suspensions or cells immobilized by κ-carragenan as a biocatalyst (Leuchtenberger et al., 2005). The initial substrate, L-aspartic acid, is notably also obtained by enzymatic conversion of ammonium fumarate (Sato et al., 1982; Leuchtenberger et al., 2005). While yields of up to 90% can be obtained, the production of fumarate is dependent on petroleum and remains costly. The cost of the L-aspartic acid intermediate is also high. More recently, enzymatic conversion of L-alanine from D-glucose and ammonium sulfate has been described (Gmelch et al., 2019). However, this method relies upon six different enzymes and production yield remains largely inferior to that obtained with immobilized P. dacunhae cells.

Fermentation by microorganisms represents an interesting alternative to the more traditional chemical and enzymatic means of generating alanine, and furthermore provides a useful way of using abundant, renewable, and/or inexpensive materials as the main source of carbon. While most microorganisms naturally produce alanine for biosynthesis (e.g. using a glutamate-pyruvate transaminase or from pyruvate and ammonia using an NADH-linked alanine dehydrogenase), fermentations are slow and yields remain low, in particular due to the production of co-products that are undesired in an industrial setting (Zhang et al., 2007). In view of improving alanine production, several microorganisms genetically modified for the production of alanine have been described to date. Such microorganisms generally overexpress AlaE (also referred to as YgaW), which is the major L-alanine exporter, and one or more heterologous genes involved in L-alanine synthesis. As an example, CN108060114 discloses an E. coli microorganism producing fumaric acid which it can then use to produce L-alanine via the enzymatic decarboxylation of L-aspartic acid described above and further comprises an overexpression of the alaE gene. WO2012172822 discloses microorganisms overexpressing both the Bacillus sphaericus gene alaD coding for an alanine dehydrogenase and the E. coli alaE gene.

Nevertheless, there remains a need for improved microorganisms that are able to produce alanine with high levels of production, titer, and yield, in particular from an inexpensive and/or abundant carbon source such as glucose. There also remains a need for novel methods for the production of alanine at a reduced cost, ideally wherein the production, titer, and/or yield of alanine is at least similar to that obtained with current methods.

### BRIEF DESCRIPTION OF THE INVENTION

The present invention concerns a microorganism genetically modified for the production of alanine and methods for the production of alanine using said microorganism. The microorganism genetically modified for the production of alanine notably expresses a heterologous alaD gene coding an alanine dehydrogenase and has reduced Lrp transcription factor activity and/or expression.

Indeed, the inventors have surprisingly found that such a microorganism shows improved production of alanine, despite the decreased expression of the alaE gene that is associated with reduced Lrp transcription factor activity and/or expression. Indeed, Lrp is known to positively regulate expression of the alaE gene (Ihara et al., 2017).

Preferably, the microorganism comprises an Irp gene coding for an Lrp* mutant having reduced transcription factor activity.

Preferably, the Lrp* mutant comprises at least one mutation selected from the group consisting of L108F, L74F, F113C, and 123PD, wherein the positions of the amino acid residues correspond to those provided in SEQ ID NO: 1.

Preferably, the microorganism comprises at least a partial deletion of the Irp gene, preferably a complete deletion of the Irp gene.

Preferably, the microorganism further comprises an overexpression of the yddG gene.

Preferably, the microorganism further comprises expression of an alaE gene coding an L-alanine exporter at a level similar to that of the corresponding microorganism which does not comprise the genetic modifications as provided herein, preferably by modifying the alaE promoter or by increasing the number of copies of the alaE gene present in the microorganism.

Preferably, the microorganism expresses the alaD gene of Geobacillus stearothermophilus, Klebsiella aerogenes, or Archaeoglobus fulgidus.

Preferably, the alaD gene codes the alanine dehydrogenase of SEQ ID NO: 15, 17, 19, 23, or 27.

Preferably, the microorganism further comprises a deletion of at least one gene selected from the group consisting of ackA-pta, IdhA, adhE, frdABCD, mgsA, and pflAB.

Preferably, the microorganism comprises the deletion of genes ackA-pta, IdhA, adhE, frdABCD, mgsA, and pflAB.

Preferably, the microorganism further comprises a deletion of the cycA and/or dadX gene(s).

Preferably, the microorganism belongs to the family of bacteria Enterobacteriaceae, Clostridiaceae, Bacillaceae, Streptomycetaceae, or Corynebacteriaceae, or to the family of yeasts Saccharomycetaceae.

Preferably, said Enterobacteriaceae bacterium is Escherichia coli or Klebsiella pneumoniae, said Clostridiaceae bacterium is Clostridium acetobutylicum, said Corynebacteriaceae bacterium is Corynebacterium glutamicum, or said Saccharomycetaceae yeast is Saccharomyces cerevisiae, more preferably said microorganism is Escherichia coli.

The present invention further comprises a method for the production of alanine comprising the steps of:
a) culturing a microorganism genetically modified for the production of alanine described in any of the embodiments provided herein in an appropriate culture medium comprising a source of carbon, and
b) recovering alanine from the culture medium.

Preferably, the source of carbon is selected from arabinose, fructose, galactose, glucose, lactose, maltose, sucrose, xylose, and any combination thereof.

### DETAILED DESCRIPTION

Before describing the present invention in detail, it is to be understood that the invention is not limited to particularly exemplified microorganisms and/or methods and may, of course, vary. It is also to be understood that the terminology used herein is for the purpose of describing particular embodiments of the invention only, and is not intended to be limiting. The invention will be limited only by the appended claims.

All publications, patents, and patent applications cited herein, whether supra or infra, are hereby incorporated by reference in their entirety. Furthermore, the practice of the present invention employs, unless otherwise indicated, conventional microbiological and molecular biological techniques that are within the skill of the art. Such techniques are well-known to the skilled person, and are fully explained in the literature. See, for example, Prescott et al. (1999) and Sambrook and Russell (2001).

Unless defined otherwise, all technical and scientific terms used herein have the same meanings as commonly understood by one of ordinary skill in the art to which this invention belongs. Although any materials and methods similar or equivalent to those described herein can be used to practice or test the present invention, preferred materials and methods are provided.

It must be noted that as used herein and in the appended claims, the singular forms “a,” “an,” and “the,” include the plural reference unless the context clearly dictates otherwise. Thus, for example, a reference to “a microorganism” includes a plurality of such microorganisms, and a reference to “an endogenous gene” is a reference to one or more endogenous genes, and so forth.

The terms “comprise,” “comprises,” and “comprising” are used in an inclusive sense, i.e. to specify the presence of the stated features but not to preclude the presence or addition of further features in various embodiments of the invention.

A first aspect of the invention relates to a microorganism genetically modified for the production of alanine.

The term “microorganism,” as used herein, refers to a living microscopic organism, which may be a single cell or a multicellular organism and which can generally be found in nature. In the present context, the microorganism is preferably a bacterium, yeast, or fungus. Preferably, the microorganism of the invention is selected from the Enterobacteriaceae, Clostridiaceae, Bacillaceae, Streptomycetaceae, or Corynebacteriaceae family or from among yeast, more preferably from the Saccharomycetaceae family. More preferably, the microorganism of the invention is a species of Escherichia, Klebsiella, Thermoanaerobacterium, Clostridium, Corynebacterium, or Saccharomyces. Even more preferably, said Enterobacteriaceae bacterium is Escherichia coli or Klebsiella pneumoniae, said Clostridiaceae bacterium is Clostridium acetobutylicum, said Corynebacteriaceae bacterium is Corynebacterium glutamicum, or said Saccharomycetaceae yeast is Saccharomyces cerevisiae. Most preferably, the microorganism of the invention is Escherichia coli.

The terms “recombinant microorganism” or “microorganism genetically modified” are used interchangeably herein and refer to a microorganism or a strain of microorganism that has been genetically modified or genetically engineered. This means, according to the usual meaning of these terms, that the microorganism of the invention is not found in nature and is genetically modified when compared to the “parental” microorganism from which it is derived. The “parental” microorganism may occur in nature (i.e. a wild-type microorganism) or may have been previously modified. The recombinant microorganism of the invention may notably be modified by the introduction, deletion and/or modification of genetic elements. Such modifications can be performed, for example, by genetic engineering, by adaptation, wherein a microorganism is cultured in conditions that apply a specific stress on the microorganism and induce mutagenesis, and/or by forcing the development and evolution of metabolic pathways by combining directed mutagenesis and evolution under specific selection pressure.

A microorganism may notably be modified to modulate the expression level of an endogenous gene or the activity of the corresponding enzyme or transcription factor. The term “endogenous gene” means that the gene was present in the microorganism before any genetic modification. Endogenous genes may be overexpressed by introducing heterologous sequences in addition to, or to replace, endogenous regulatory elements. Endogenous gene expression levels, protein expression levels, or the activity of the encoded protein, can also be increased or attenuated by introducing mutations into the coding sequence of a gene or into non-coding sequences. These mutations may be synonymous, when no modification in the corresponding amino acid occurs, or non-synonymous, when the corresponding amino acid is altered. Synonymous mutations do not have any impact on the function of translated proteins, but may have an impact on the regulation of the corresponding genes or even of other genes, if the mutated sequence is located in a binding site for a regulator factor. Non-synonymous mutations may have an impact on the function or activity of the translated protein as well as on regulation depending on the nature of the mutated sequence.

In particular, mutations in non-coding sequences may be located upstream of the coding sequence (i.e. in the promoter region, in an enhancer, silencer, or insulator region, in a specific transcription factor binding site) or downstream of the coding sequence. Mutations introduced in the promoter region may be in the core promoter, proximal promoter, or distal promoter. Mutations may be introduced by site-directed mutagenesis using, e.g., Polymerase Chain Reaction (PCR), by random mutagenesis techniques e.g. via mutagenic agents (Ultra-Violet rays or chemical agents like nitrosoguanidine (NTG) or ethylmethanesulfonate (EMS)), DNA shuffling, error-prone PCR, or using culture conditions that apply a specific stress on the microorganism and induce mutagenesis. The insertion of one or more supplementary nucleotide(s) in the region located upstream of a gene can notably modulate gene expression.

A particular way of modulating endogenous gene expression is to exchange the endogenous promoter of a gene (e.g., wild-type promoter) with a stronger or weaker promoter to upregulate or downregulate expression of the endogenous gene. The promoter may be endogenous (i.e. originating from the same species) or exogenous (i.e. originating from a different species). It is well within the ability of the person skilled in the art to select an appropriate promoter for modulating the expression of an endogenous gene. Such a promoter may be, for example, a Ptrc, Ptac, or Plac promoter, or the PR or PL lambda promoters. The promoters may be “inducible” by a particular compound or by specific external conditions, such as temperature or light.

A particular way of modulating endogenous protein activity is to introduce nonsynonymous mutations in the coding sequence of the corresponding gene, e.g. according to any of the methods described above. A non-synonymous amino acid mutation that is present in a transcription factor may notably alter binding affinity of the transcription factor toward a cis-element, alter ligand binding to the transcription factor, etc.

A microorganism may also be genetically modified to express one or more exogenous (i.e. heterologous) genes so as to express or overexpress the corresponding gene product (e.g. an enzyme). An “exogenous” or “heterologous” gene as used herein refers to a gene encoding a protein or polypeptide that is introduced into a microorganism in which said gene does not naturally occur. A “heterologous gene” as used herein also refers to a gene that was endogenous to a microorganism (i.e. present in the microorganism prior to any genetic modification) but that, when introduced into the microorganism, is not introduced at the location where the endogenous gene is/was located. More particularly, the heterologous gene may be an endogenous gene in cases where expression of endogenous gene itself in the microorganism is reduced as compared to the microorganism in which the gene naturally occurs (e.g. due to a mutation, a complete or partial deletion of the gene, a modification in the transcriptional regulation of the gene, etc.). In particular, the endogenous gene may no longer be expressed or may be expressed at very low levels. The exogenous gene may be directly integrated into the chromosome of the microorganism, or be expressed extra-chromosomally within the microorganism by plasmids or vectors. For successful expression, exogenous gene(s) must be introduced into the microorganism with all of the regulatory elements necessary for their expression or be introduced into a microorganism that already comprises all of the regulatory elements necessary for their expression. The genetic modification or transformation of microorganisms with one or more exogenous genes is a routine task for those skilled in the art.

One or more copies of a given exogenous gene can be introduced on a chromosome by methods well-known in the art, such as by genetic recombination. When a gene is expressed extra-chromosomally, it can be carried by a plasmid or a vector. Different types of plasmid are notably available, which may differ in respect to their origin of replication and/or their copy number in the cell. For example, a microorganism transformed by a plasmid can contain 1 to 5 copies of the plasmid, about 20 copies, or even up to 500 copies, depending on the nature of the selected plasmid. A variety of plasmids having different origins of replication and/or copy numbers are well-known in the art and can be easily selected by the skilled person for such purposes, including, e.g., pTrc, pACYC184, pBR322, pUC18, pUC19, pKC30, pRep4, pHS1, pHS2, or pPLc236.

It should be understood that, in the context of the present invention, when an exogenous gene encoding a protein of interest is expressed in a microorganism, a synthetic version of this gene may preferably be constructed by replacing non-preferred codons or less preferred codons with preferred codons of said microorganism which encode the same amino acid. Indeed, it is well-known in the art that codon usage varies between microorganism species, and that this may impact the recombinant expression level of the protein of interest. To overcome this issue, codon optimization methods have been developed, and are extensively described by Graf et al. (2000), Deml et al. (2001) and Davis & Olsen (2011). Several software programs have notably been developed for codon optimization determination such as the GeneOptimizer® software (Lifetechnologies) or the OptimumGene™ software of (GenScript). In other words, the exogenous gene encoding a protein of interest is preferably codon-optimized for expression in the microorganism. As a particular example, the heterologous alaD gene may be codon optimized for expression in a microorganism such as E. coli.

The terms “expressing,” “overexpressing,” or “overexpression” of a protein of interest, such as an enzyme, refer herein to an increase in the expression level and/or activity of said protein in a microorganism, as compared to the corresponding parent microorganism that does not comprise the modification(s) present in the genetically modified microorganism. In some cases, the level of expression may be similar to that of the parent microorganism. In other cases, the level of expression may be superior to that of the parent microorganism. In cases where a parent microorganism does not comprise the protein of interest, the term “expression” or “overexpression” refers to the presence of the protein of interest, as compared to its absence in the parent microorganism.

In contrast, the terms “attenuating” or “attenuation” of a protein of interest refer to a decrease in the expression level and/or activity of said protein in a microorganism, as compared to the parent microorganism. The attenuation of expression can notably be due to either the exchange of the wild-type promoter for a weaker natural or synthetic promoter or the use of an agent reducing gene expression, such as antisense RNA or interfering RNA (RNAi), and more particularly small interfering RNAs (siRNAs) or short hairpin RNAs (shRNAs). Promoter exchange may notably be achieved by the technique of homologous recombination (Datsenko & Wanner, 2000). The complete attenuation of the expression level and/or activity of a protein of interest means that expression and/or activity is abolished; thus, the expression level of said protein is null. The complete attenuation of the expression level and/or activity of a protein of interest may be due to the complete suppression of the expression of a gene. This suppression can be either an inhibition of the expression of the gene, a deletion of all or part of the promoter region necessary for expression of the gene, or a deletion of all or part of the coding region of the gene. A deleted gene can notably be replaced by a selection marker gene that facilitates the identification, isolation, and purification of the modified microorganism. As a non-limiting example, suppression of gene expression may be achieved by the technique of homologous recombination, which is well-known to the person skilled in the art (Datsenko & Wanner, 2000).

Modulating the expression level of one or more proteins may thus occur by altering the expression of one or more endogenous genes that encode said protein within the microorganism as described above or by introducing one or more heterologous genes that encode said protein into the microorganism.

The term “expression level” as used herein, refers to the amount (e.g. relative amount, concentration) of a protein of interest (or of the gene encoding said protein) expressed in a microorganism, which is measurable by methods well-known in the art. The level of gene expression can be measured by various known methods including Northern blotting, quantitative RT-PCR, and the like. Alternatively, the level of expression of the protein coded by said gene may be measured, for example by SDS-PAGE, HPLC, LC/MS, and other quantitative proteomic techniques (Bantscheff et al., 2007), or, when antibodies against said protein are available, by Western Blot-Immunoblot (Burnette, 1981), Enzyme-linked immunosorbent assay (e.g. ELISA) (Engvall and Perlman, 1971), protein immunoprecipitation, immunoelectrophoresis, and the like. The copy number of an expressed gene can be quantified, for example, by restricting chromosomal DNA followed by Southern blotting using a probe based on the gene sequence, fluorescence in situ hybridization (FISH), RT-qPCR, and the like.

Overexpression of a given gene or the corresponding protein may be verified by comparing the expression level of said gene or protein in the genetically modified organism to the expression level of the same gene or protein in a control microorganism that does not have the genetic modification (i.e. the parental microorganism).

The terms “activity” or “function” as used herein in the context of an enzyme designate the reaction that is catalyzed by said enzyme for converting its corresponding substrate(s) into another molecule(s) (i.e. product(s)). As is well-known in the art, the activity of an enzyme may be assessed by measuring its catalytic efficiency and/or Michaelis constant. Such an assessment is described for example in Segel, 1993, in particular on pages 44-54 and 100-112, incorporated herein by reference.

The term “transcription factor” as used herein refers to a protein, more specifically the “leucine regulatory protein” or “Lrp,” that possesses a biological function including regulation of transcription of genes. The Lrp transcription factor possesses a DNA-binding domain that allows it to bind a specific sequence of DNA such as an enhancer element or promoter sequence. Binding may in some cases be dependent on the presence or absence of an allosteric binding protein, such as leucine. Upon binding the enhancer or promoter element, the transcription factor may aide in initiation of transcription, for example, by stabilizing transcription initiation complex formation and/or activity. Transcription factors may also bind to regulatory DNA sequences, such as enhancer sequences, that may be many hundreds of base pairs downstream or upstream from the transcribed gene. Transcription factors may modulate transcription either alone or in combination with other proteins, i.e. by forming an activation complex that may aide in recruiting RNA polymerase and related proteins to the transcription initiation start site.

“Transcription factor activity” as used herein refers to the capacity of a transcription factor to modulate expression of one or more genes by increasing or decreasing the rate of their transcription. The transcription factor may act directly on the gene, e.g. by binding a cis element present in the promoter of the gene or indirectly, e.g. by modulating expression of another element or transcription factor which in turn regulates the transcription of the gene. Transcription factor activity may be evaluated, for example, by electrophoretic gel shift assay or DNA footprinting in cases where the transcription factor directly binds to a cis-element. Reporter assays using e.g. β-galactosidase, luciferase, or GFP (Green Fluorescent Protein) as a reporter gene may also be used to determine transcription factor activity. More particularly, in the context of the present invention, Lrp transcription factor activity and/or expression is reduced as compared to that of a parent microorganism. In turn, the inventors have found that this surprisingly reduces alaE gene expression (data not shown). In other words, the microorganism provided herein notably has reduced alaE gene expression as compared to that of the parent microorganism. Preferably, Lrp transcription factor activity and/or expression is reduced as compared to the transcription factor activity and/or expression level of the Lrp protein of SEQ ID NO: 1.

The microorganism genetically modified for the production of alanine provided herein expresses a heterologous gene coding an enzyme having alanine dehydrogenase activity and has reduced Lrp transcription factor activity and/or expression. Indeed, the inventors have surprisingly shown that the above genetic modifications improve alanine titer, production, and yield, as compared to a parent microorganism that does not comprise these modifications. Improved alanine production in this microorganism is particularly surprising as reduced Lrp transcription factor activity and/or expression reduces the expression level of the alaE gene coding the L-alanine exporter. Indeed, alaE is generally overexpressed in microorganisms modified for the production of L-alanine.

Preferably, the microorganism genetically modified for the production of alanine provided herein expresses a heterologous alaD gene coding an alanine dehydrogenase and has reduced Lrp transcription factor activity and/or expression. More specifically, the microorganism genetically modified for the production of alanine provided herein expresses a heterologous alaD gene coding an alanine dehydrogenase and has reduced Lrp transcription factor activity and/or expression as compared to a parent microorganism (e.g. as compared to the Lrp transcription factor activity and/or expression in the corresponding wild-type microorganism a wild-type microorganism).

Preferably, the Lrp protein itself is attenuated. Preferably, the microorganism of the invention comprises an Irp gene coding for a mutated Lrp protein (otherwise referred to herein as an “Lrp* mutant” protein) having reduced transcription factor activity. The skilled person may readily determine if a given Lrp* protein has reduced transcription factor activity.

As a non-limiting example, the Lrp* mutant may comprise one, two, three or more amino acid substitutions and one, two or more amino acid insertions. When an amino acid in a protein is replaced by another amino acid, the total number of amino acids in the protein does not change. In contrast, when one or more amino acids are inserted, the number of amino acids in the protein increases accordingly. The position of an insertion is the position at which the amino acid(s) are inserted with respect to the unmodified sequence (i.e. the corresponding position).

Corresponding positions can notably be determined by those skilled in the art using manual alignment or by using an alignment program (e.g., BLASTP). Corresponding positions can also be based on structural alignments, for example by using computer-simulated alignments of protein structures. The fact that an amino acid of a polypeptide corresponds to an amino acid in the disclosed sequence means that when the polypeptide and the disclosed sequence are aligned, a standard alignment calculation method such as a GAP calculation method is used. A corresponding amino acid may notably be identified when conserved amino acids are aligned such that the sequences have maximized identity or homology. As used herein, “in a corresponding position” refers to a position of interest in a nucleic acid molecule or protein (i.e. nucleotide base or amino acid residue number) relative to a position in a reference nucleic acid molecule or protein. Positions of interest relative to positions in reference proteins can be, for example, allelic variants, heterologous proteins, amino acid sequences of the same protein in other species, etc. Corresponding positions can be determined by comparing and aligning sequences such that the number of paired nucleotides or amino acid residues is maximized. For example, identity between sequences may be greater than 95%, 96%, 97%, 98%, or more particularly greater than 99%. The position of interest is then given the number assigned in the sequence of the reference nucleic acid molecule or polypeptide. The skilled person will recognize that, in an Lrp* mutant polypeptide, amino acid residue 1 of the modified polypeptide corresponds to amino acid residue 1 of the unmodified Lrp polypeptide (as provided in SEQ ID NO: 1). Indeed, SEQ ID NO: 1 as provided herein is the reference sequence for the Lrp protein. Similarly, SEQ ID NO: 2 as provided herein is the reference sequence for the Irp gene.

Preferably, said amino acid mutations are located in the protein C-terminal domain corresponding to the RAM (regulation of amino-acid metabolism) domain. Said substitutions may notably be selected from among L108F, L74F, and F113C. The positions of the amino acid residues indicated correspond to those provided in SEQ ID NO: 1. In the case of L108F and L74F, the substituting amino acid is not limited to phenylalanine but may be any amino acid with an uncharged residue. As a non-limiting example, instead of phenylalanine, L108 and/or L74 may be replaced with tyrosine, tryptophan, alanine, isoleucine, or valine. Said insertion may correspond to 123PD. In the context of the present invention, the insertion “123PD” corresponds to an insertion of two amino acids after position 123. In other words, the amino acids at positions 124 and 125 of the corresponding Lrp* are 124P and 125D, the amino acids at or after position 124 of SEQ ID NO: 1 have been displaced by two amino acid residues.

According to a preferred embodiment, the microorganism comprises an Lrp* mutant wherein the Lrp* mutant comprises at least one mutation selected from the group consisting of L108F, L74F, F113C, and 123PD, wherein the positions of the amino acid residues correspond to those provided in SEQ ID NO: 1. Thus, according to a preferred embodiment, the microorganism comprises an Lrp* mutant having the sequence of SEQ ID NO: 3, 5, 7, or 9. According to a preferred embodiment, the microorganism comprises a gene coding for an Lrp* mutant having the sequence of SEQ ID NO: 4, 6, 8, or 10.

Alternatively, the microorganism comprises an attenuation in the expression of the Irp gene. More particularly, said attenuation results from at least a partial deletion of the Irp gene or a complete deletion. The term “partial deletion” as used herein refers to the loss of at least 0.5%, 1%, 5%, 10%, 20%, 40%, 50%, 60%, 70%, 80%, 90%, or 99% of the nucleotides forming the nucleotide sequence of a gene. The term “complete deletion” as used herein refers to the loss of 100% of the nucleotides forming the nucleotide sequence of a gene.

According to a preferred embodiment, the microorganism comprises at least a partial deletion of the Irp gene, more preferably a complete deletion of the Irp gene.

As mentioned above, the microorganism comprises a heterologous enzyme having alanine dehydrogenase activity. Preferably, said enzyme having alanine dehydrogenase activity is encoded by a heterologous alaD gene. Said heterologous enzyme having alanine dehydrogenase activity or said heterologous alaD gene coding an alanine dehydrogenase may be derived from a microorganism of one of the following genera: Bacillus, Geobacillus, Klebsiella, Archaeoglobus, Lysinibacillus, Thermus, Mycobacterium, or Phormidium. More particularly, said Bacillus may be Bacillus subtilis, said Geobacillus may be Geobacillus stearothermophilus, said Klebsiella may be Klebsiella aerogenes, said Archaeoglobus may be Archaeoglobus fulgidus, said Lysinibacillus may be Lysinibacillus sphaericus, said Thermus may be Thermus thermophilus, said Mycobacterium may be Mycobacterium tuberculosis, or said Phormidium may be Phormidium lapideum. According to a preferred embodiment, the microorganism expresses an alaD gene of Geobacillus stearothermophilus, Klebsiella aerogenes, or Archaeoglobus fulgidus. According to a preferred embodiment, the microorganism expresses an alaD gene coding for an alanine dehydrogenase of SEQ ID NO: 11, 13, 15, 17, 19, 21, 23, 25, 27 or 29, or a functional fragment or functional variant thereof. More preferably, the alaD gene codes an alanine dehydrogenase of SEQ ID NO: 11, 13, 15, 17, 19, 21, 23, 25, 27 or 29, even more preferably, the alaD gene codes the alanine dehydrogenase of SEQ ID NO: 15, 17, 19, 23, or 27. Preferably, said heterologous alaD gene coding an alanine dehydrogenase is selected from among the genes having the sequence of SEQ ID NO: 12, 14, 16, 18, 20, 22, 24, 26, 28 and 30. More preferably, said heterologous alaD gene coding an alanine dehydrogenase is selected from among the genes having the sequence of SEQ ID NO: 16, 18, 20, 24, and 28.

The term “functional fragment” of a protein of reference having a biological activity of interest (e.g. of an enzyme having alanine dehydrogenase activity), as used herein refers to parts of the amino acid sequence of an enzyme, said parts comprising at least all the regions essential for exhibiting the biological activity of said protein. These parts of sequences can be of various lengths, provided that the biological activity of the amino acid sequence of reference is retained by said parts. In other words, the functional fragments of the enzymes provided herein are enzymatically active.

“Functional variants” of an enzyme described herein (e.g. of an enzyme having alanine dehydrogenase activity) include, but are not limited to, enzymes having amino acid sequences which are at least 60% identical after alignment to the amino acid sequence encoding the corresponding reference enzyme. According to the present invention, the variant preferably has at least 70%, 80%, 85%, 90%, 95%, 96%, 97%, 98%, 99%, or 100% amino acid sequence identity to the protein described herein (e.g. an AlaD protein). Thus, the enzyme having alanine dehydrogenase activity preferably has at least 70%, 80%, 85%, 90%, 95%, 96%, 97%, 98%, 99%, or 100% sequence identity to the amino acid sequence of SEQ ID NO: 11, 13, 15, 17, 19, 21, 23, 25, 27, or 29. More preferably, the gene encoding the enzyme having alanine dehydrogenase activity has at least 70%, 80%, 85%, 90%, 95%, 96%, 97%, 98%, 99%, or 100% sequence identity to the nucleotide sequence of SEQ ID NO: 12, 14, 16, 18, 20, 22, 24, 26, 28, or 30. As a non-limiting example, means of determining sequence identity are further provided below.

In addition to the modifications described above, the genetically modified microorganism may comprise one or more additional modifications among those described below. Said modifications are advantageous as they may notably further improve alanine production, titer, and/or yield. One or more of said modifications may notably promote alanine synthesis, inhibit the use of alanine as a substrate in downstream metabolic pathways, promote stable accumulation of alanine, or inhibit toxic accumulation of alanine in the microorganism.

In particular, said microorganism may further comprise the overexpression of the aromatic amino acid exporter YddG. Preferably, the amino acid sequence YddG has at least 80% identity, more preferably at least 90% identity, even more preferably at least 95%, 96%, 97%, 98%, or 99% identity, most preferably 100% identity, with the sequence of SEQ ID NO: 31. YddG may notably be overexpressed via the introduction of an exogenous yddG gene into the microorganism (e.g. on a plasmid). Alternatively or in addition, the endogenous yddG gene may be overexpressed by one or more modifications to said gene or corresponding promoter region, preferably as described herein. Preferably, the yddG gene coding for said exporter is overexpressed. Preferably, said yddG gene encodes the YddG protein having the sequence of SEQ ID NO: 31. More preferably, said yddG gene has a sequence having at least 80% sequence identity, more preferably at least 90% identity, even more preferably at least 95%, 96%, 97%, 98%, or 99% identity, most preferably 100% identity with the sequence of SEQ ID NO: 32.

According to a preferred embodiment, the microorganism further comprises an overexpression of the yddG gene, more preferably wherein a plasmid comprising a yddG gene is introduced into said microorganism.

The microorganism may further comprise expression of the L-alanine exporter AlaE. Indeed, in the microorganism of the invention, AlaE expression is significantly inhibited due to reduced Lrp transcription factor activity and/or expression. AlaE expression is preferably inferior or equal to that present in the corresponding parent microorganism (i.e. which does not comprise the genetic modifications described herein, in particular which does not comprise reduced Lrp transcription factor activity and/or expression and which does not comprise a heterologous alaD gene coding an alanine dehydrogenase according to any of the embodiments provided herein). In other words, AlaE expression is at least partially, preferably completely, restored to levels observed in the corresponding parent microorganism. Preferably the level of AlaE expression is similar to that of the corresponding parent microorganism. Preferably the level of AlaE expression is not superior to that of the corresponding parent microorganism. Preferably, the amino acid sequence of AlaE has at least 80% identity, more preferably at least 90% identity, even more preferably at least 95% identity, most preferably 100% identity, with the sequence of SEQ ID NO: 33.

AlaE expression may notably be restored by modifying the alaE promoter or by increasing the number of copies of the alaE gene present in the microorganism, in particular according to one of the methods described herein.

Thus, according to a preferred embodiment, the microorganism further comprises expression of an alaE gene coding an L-alanine exporter at a level similar to that of the corresponding parent microorganism, preferably by modifying the alaE promoter or by increasing the number of copies of the alaE gene present in the microorganism. Preferably, the alaE gene has at least 80% identity, more preferably at least 90% identity, even more preferably at least 95% identity, most preferably 100% identity, with the sequence of SEQ ID NO: 34.

According to a particularly preferred embodiment, the microorganism provided herein further comprises both an overexpression of the yddG gene and expression of an alaE gene coding an L-alanine exporter at a level similar to that of the corresponding parent microorganism, preferably by modifying the alaE promoter or by increasing the number of copies of the alaE gene present in the microorganism.

The microorganism may further comprise the attenuation of at least one enzyme selected from among the acetate kinase AckA, the aldehyde-alcohol dehydrogenase AdhE, the fumarate reductase FrdABCD, comprising a flavoprotein subunit FrdA, a fumarate reductase iron-sulfur protein FrdB, and the fumarate reductase membrane proteins FrdC and FrdD, the D-lactate dehydrogenase LdhA, the methylglyoxal reductase MgsA, the pyruvate formate-lyase activating enzyme PflA, the inactive pyruvate formate-lyase PfIB, and the phosphate acetyltransferase Pta. Preferably, when the activity of at least one of the above enzymes is attenuated, said activity is completely attenuated. Said complete attenuation is preferably due to a partial or complete deletion of the gene coding for said enzyme, even more preferably a complete deletion of the gene coding for said enzyme.

Thus, according to a preferred embodiment of the invention, the microorganism further comprises the deletion of at least one of the following genes: ackA, adhE, frdABCD, IdhA, mgsA, pflAB, and pta, or any combination thereof. More preferably, the microorganism further comprises the deletion of the genes: ackA, adhE, frdABCD, IdhA, mgsA, pflAB, and pta. Said genes are notably endogenous in E. coli. Preferably, said ackA-pta genes have the sequence having at least 80% sequence identity, more preferably at least 90% identity, even more preferably at least 95% identity, most preferably 100% identity, with the sequence of SEQ ID NO: 35 and the sequence of SEQ ID NO: 36, respectively. Preferably, said adhE gene has a sequence having at least 80% sequence identity, more preferably at least 90% identity, even more preferably at least 95% identity, most preferably 100% identity, with the sequence of SEQ ID NO: 37. Preferably, said frdABCD genes have the sequences having at least 80% sequence identity, more preferably at least 90% identity, even more preferably at least 95% identity, most preferably 100% identity, with the sequences of SEQ ID NOs: 38, 39, 40, and 41, respectively. Preferably, said IdhA gene has a sequence having at least 80% sequence identity, more preferably at least 90% identity, even more preferably at least 95% identity, most preferably 100% identity, with the sequence of SEQ ID NO: 42. Preferably, said mgsA gene has a sequence having at least 80% sequence identity, more preferably at least 90% identity, even more preferably at least 95% identity, most preferably 100% identity, with the sequence of SEQ ID NO: 43. Preferably, said pflAB genes have the sequences having at least 80% sequence identity, more preferably at least 90% identity, even more preferably at least 95% identity, most preferably 100% identity, with the sequences of SEQ ID NOs: 44 and 45. Preferably, said deletion is a complete deletion of the coding region of each of said genes.

Preferably, the microorganism comprises an attenuation of the inner membrane protein CycA which mediates the uptake of D-serine, D-alanine, and glycine and/or the DadX alanine racemase. Said genes are notably endogenous in E. coli. Preferably, expression of CycA and/or DadX is attenuated, more preferably completely attenuated. Preferably, the cycA and/or dadX gene is attenuated, preferably due to a partial or complete deletion of the gene(s) coding for said protein(s), more preferably a complete deletion of the gene(s). Preferably, the cycA gene has a sequence having at least 80% sequence identity, more preferably at least 90% identity, even more preferably at least 95% identity, most preferably 100% identity, with the sequence of SEQ ID NO: 46. Preferably, the dadX gene has a sequence having at least 80% sequence identity, more preferably at least 90% identity, even more preferably at least 95% identity, most preferably 100% identity, with the sequence of SEQ ID NO: 47.

In a further aspect, when the microorganism as described herein is unable to use sucrose as a carbon source, said microorganism is modified to be able to use sucrose as a carbon source. Preferably, proteins involved in the import and metabolism of sucrose are overexpressed. Preferably, the following proteins are overexpressed:
CscB sucrose permease, CscA sucrose hydrolase, CscK fructokinase, and CscR csc-specific repressor, or
ScrA Enzyme II of the phosphoenolpyruvate-dependent phosphotransferase system, said ScrK gene encodes ATP-dependent fructokinase, ScrB sucrose 6-phosphate hydrolase (invertase), ScrY sucrose porine, and ScrR sucrose operon repressor.

Preferably, genes coding for said proteins are overexpressed according to one of the methods provided herein. Preferably, the E. coli microorganism overexpresses:
the heterologous cscBKAR genes of E. coli EC3132, or
the heterologous scrKYABR genes of Salmonella sp.

Genes and proteins are identified herein using the denominations of the corresponding genes in E. coli (e.g. E. coli K12 MG1655 having the Genbank accession number U00096.3) unless otherwise specified. However, in some cases use of these denominations has a more general meaning according to the invention and covers all of the corresponding genes and proteins in microorganisms. This is notably the case for the genes and proteins described herein that are not endogenous to the microorganism of the invention (i.e. that are heterologous), such as AlaD. As a particular example, and as indicated above, functional variants of AlaD, are comprised herein, as are mutants and functional fragments thereof. Particular aspects are further detailed below.

PFAM (protein family database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gov/COG/) are obtained by comparing protein sequences from 43 fully sequenced genomes representing 30 major phylogenic lines. Each COG is defined from at least three lines, which permits the identification of former conserved domains.

The means of identifying similar sequences and their percent identities are well-known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given on GenBank for known genes, the person skilled in the art is able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well-known to those skilled in the art, and are described, e.g., in Sambrook and Russell, 2001.

Sequence identity between amino acid sequences can be determined by comparing a position in each of the sequences which may be aligned for the purposes of comparison. When a position in the compared sequences is occupied by the same amino acid, then the sequences are identical at that position. A degree of sequence identity between proteins is a function of the number of identical amino acid residues at positions shared by the sequences of said proteins.

As a non-limiting example, to determine the percentage of identity between two amino acid sequences, the sequences are aligned for optimal comparison. For example, gaps can be introduced in the sequence of a first amino acid sequence for optimal alignment with the second amino acid sequence. The amino acid residues at corresponding amino acid positions are then compared. When a position in the first sequence is occupied by the same amino acid residue as the corresponding position in the second sequence, the molecules are identical at that position.

The percentage of identity between the two sequences is a function of the number of identical positions shared by the sequences. Hence % identity = number of identical positions /-total number of overlapping positions _{X} 100.

Optimal alignment of sequences may be conducted by the global alignment algorithm of Needleman and Wunsch (1972), by computerized implementations of this algorithm (such as CLUSTAL W) or by visual inspection. The best alignment (i.e., resulting in the highest percentage of identity between the compared sequences) generated by the various methods is selected.

In other words, the percentage of sequence identity is calculated by comparing two optimally aligned sequences, determining the number of positions at which the identical amino acid occurs in both sequences to yield the number of matched positions, dividing the number of matched positions by the total number of positions and multiplying the result by 100 to yield the percentage of sequence identity.

The above definitions and preferred embodiments related to the functional fragments and functional variants of proteins apply mutatis mutandis to nucleotide sequences, such as genes, encoding a protein of interest (i.e. an enzyme having alanine dehydrogenase activity).

A second object of the invention relates to a method for the production of alanine using the microorganism described herein. Said method comprises the steps of:
a) culturing a microorganism genetically modified for the production of alanine as described herein in an appropriate culture medium comprising a source of carbon, and
b) recovering alanine from the culture medium.

More specifically, the invention relates to a method for the improved fermentative production of alanine using the microorganism described herein. According to the invention, the terms “fermentative process,” “fermentative production,” “fermentation,” or “culture” are used interchangeably to denote the growth of microorganism. This growth is generally conducted in fermenters with an appropriate growth medium adapted to the microorganism being used.

An “appropriate culture medium” designates a medium (e.g., a sterile, liquid media) comprising nutrients essential or beneficial to the maintenance and/or growth of the cell such as carbon sources or carbon substrates, nitrogen sources, for example, peptone, yeast extracts, meat extracts, malt extracts, urea, ammonium sulfate, ammonium chloride, ammonium nitrate, and ammonium phosphate; phosphorus sources, for example, monopotassium phosphate or dipotassium phosphate; trace elements (e.g., metal salts), for example magnesium salts, cobalt salts, and/or manganese salts; as well as growth factors such as amino acids and vitamins. In particular, the inorganic culture medium for E. coli can be of identical or similar composition to an M9 medium (Anderson, 1946), an M63 medium (Miller, 1992), or a medium such as defined by Schaefer et al. (1999).

The term “source of carbon,” “carbon source,” or “carbon substrate” according to the present invention refers to any carbon source capable of being metabolized by a microorganism wherein the substrate contains at least one carbon atom. According to the present invention, said source of carbon is preferably at least one carbohydrate, and in some cases a mixture of at least two carbohydrates. CO₂ is not a carbohydrate because it does not contain hydrogen.

The term “carbohydrate” refers to any carbon source capable of being metabolized by a microorganism and containing at least one carbon atom, two atoms of hydrogen and one atom of oxygen. The one or more carbohydrates may be selected from among the group consisting of: monosaccharides such as glucose, fructose, mannose, xylose, arabinose, galactose, and the like, disaccharides such as sucrose, cellobiose, maltose, lactose, and the like, oligosaccharides such as raffinose, stacchyose, maltodextrins, and the like, polysaccharides such as cellulose, hemicellulose, starch, and the like, methanol, formaldehyde, and glycerol. Preferred carbon sources are arabinose, fructose, galactose, glucose, lactose, maltose, sucrose, xylose, or any combination thereof, more preferably glucose.

The term “recovering” as used herein designates the process of separating or isolating the produced alanine by using conventional laboratory techniques known to the person skilled in the art. Recovering alanine according to step b) of the method described herein may comprise a step of filtration, desalination, cation exchange, liquid extraction, crystallization, or distillation, or combinations thereof. Alanine may be recovered from both culture medium and microorganisms, or from only one or the other. Preferably, alanine is recovered from at least the culture medium. The volume of culture medium may be reduced for example via ceramic membrane filtration. Alanine may furthermore be recovered either during culturing of the microorganism by in situ product recovery including extractive fermentation, or after fermentation is finished. Microorganisms may notably be removed by passing through a device, preferably through a filter with a cut-off in the range from 5 to 200 kDa, where solid/liquid separation takes place. It is also feasible to employ a centrifuge, a suitable sedimentation device, or a combination of these devices, it being especially preferred to first separate at least part of the microorganisms by sedimentation and subsequently to feed the fermentation broth, from which the microorganisms have been at least partially removed, to ultrafiltration or to a centrifugation device. After the microorganisms have been removed, alanine present in the remaining culture medium may be recovered. Alanine may be recovered from microorganisms separately. Recovery of alanine from microorganism may notably involve lysis or disruption by heating to induce alanine release from microorganisms.

Those skilled in the art are able to define the culture conditions for the microorganisms according to the invention. In particular the bacteria are fermented at a temperature between 20° C. and 55° C., preferably between 25° C. and 40° C., more preferably between about 30° C. to 37° C., even more preferably about 37° C.

This process can be carried out either in a batch process, in a fed-batch process, or in a continuous process. It can be carried out under aerobic, micro-aerobic, or anaerobic conditions, or a combination thereof (for example, aerobic conditions followed by anaerobic conditions).

“Under aerobic conditions” means that oxygen is provided to the culture by dissolving the gas into the liquid phase. This could be obtained by (1) sparging oxygen containing gas (e.g. air) into the liquid phase or (2) shaking the vessel containing the culture medium in order to transfer the oxygen contained in the head space into the liquid phase. The main advantage of the fermentation under aerobic conditions is that the presence of oxygen as an electron acceptor improves the capacity of the strain to produce more energy under the form of ATP for cellular processes. Therefore, the strain has its general metabolism improved.

Micro-aerobic conditions are defined as culture conditions wherein low percentages of oxygen (e.g. using a mixture of gas containing between 0.1 and 10% oxygen, completed to 100% with nitrogen), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The production of alanine by the microorganism in the culture broth can be determined unambiguously by standard analytical means known by those skilled in the art. As a non-limiting example, alanine may be quantified using isocratic HPLC (Pleissner et al., 2011) or nuclear magnetic resonance.

In a further aspect, the method described herein further comprises a step c) of purifying alanine. Alanine may be purified by using conventional laboratory techniques known to the skilled person, such as concentration, filtration, ion-exchange, or crystallization methods, or combinations thereof. Alanine may be further purified by using conventional laboratory techniques known to the skilled person, such as filtration and/or crystallization. Methods of recovering and/or purifying alanine are notably described in CN103965064A and CN103965064A. As an example, alanine may be purified after being mixed with an organic solvent.

### EXAMPLES

The present invention is further defined in the following examples. It should be understood that these examples, while indicating preferred embodiments of the invention, are given by way of illustration only. From above disclosure and these examples, the person skilled in the art can make various changes to the invention to adapt it to various uses and conditions without modifying the essential means of the invention.

In the examples given below, methods well-known in the art were used to construct E. coli strains containing replicating vectors and/or various chromosomal deletions, and substitutions using homologous recombination, as is well-described in Datsenko & Wanner, (2000) for E. coli. In the same manner, the use of plasmids or vectors to express or overexpress one or more genes in a recombinant microorganism are well-known by the person skilled in the art. Examples of suitable E. coli expression vectors include pTrc, pACYC184, pBR322, pUC18, pUC19, pKC30, pRep4, pHS1, pHS2, pPLc236, etc.

Chromosomal modifications. Several protocols have been used in the following examples. Protocol 1 (chromosomal modifications by PCR amplification using oligonucleotides and appropriate genomic DNA as a matrix (that the person skilled in the art will be able to define), homologous recombination, and selection of recombinants), protocol 2 (transduction of phage P1) and protocol 3 (antibiotic cassette excision, the resistance genes were removed when necessary) used in this invention have been fully described in patent application EP 2532751 (see in particular example 1 and example 3, points 1.2 and 1.3, incorporated herein by reference). Chromosomal modifications were verified by PCR analysis with appropriate oligonucleotides that the person skilled in the art is able to design.

Construction of recombinant plasmids. Recombinant DNA technology is well described in the literature and routinely used by the person skilled in the art. Briefly, DNA fragments were PCR amplified using oligonucleotides and appropriate genomic DNA as a matrix (that the person skilled in the art will be able to define). The DNA fragments and chosen plasmid were digested with compatible restriction enzymes (that the person skilled in the art will be able to define), then ligated and transformed into competent cells. Transformants were analyzed and recombinant plasmids of interest were verified by DNA sequencing. Kanamycin (50 mg/L) and/or chloramphenicol (30 mg/L) was added to the medium when necessary.

Strain selection based on the expression of genes responsible for antibiotic resistance. Strains construction required the selection of cells harboring a DNA fragment responsible for a specific antibiotic resistance. To achieve this selection, bacteria were spread on petri dishes containing LB solid medium (10 g/L bactopeptone, 5 g/L yeast extract, 5 g/L NaCl and 20 g/L agar). Antibiotics were added when necessary according to the selection marker: chloramphenicol (30 mg/L); kanamycin (50 mg/L); gentamycin (10 mg/L).

Culture conditions. Strains that produced substantial amounts of metabolites of interest were subsequently tested under production conditions in 2.5 L fermentors (Pierre Guerin) using a strategy of limiting growth by the feeding rate and glucose concentration in the feeding medium. A 50 mL preculture was grown at 37° C. for 16 hours in a mixed medium (10% LB medium with 2.5 g.L⁻¹ glucose and 90% B1 minimal medium, Table 1). It was used to inoculate a 1 L culture to an OD₆₀₀ of 1 in PC1 medium. The culture temperature was maintained constant at 37° C. and pH was maintained to the working value (6.8) by automatic addition of NH₄OH solution (28% NH₄OH). The initial agitation rate was set at 150 RPM. After 6 hours of batch phase, the feeding rate (F1 medium) was started at a value of 0.01 L.h⁻¹ and kept constant for 30 hours. Then, the feeding rate was gradually increased to a value of 0.05 L.h⁻¹. Glucose was limiting for the duration of the culture. Strains were compared after culture for 120 hours.

TABLE 1

Media composition for the culture of alanine production strains.

| Compound | Concentration (g/L) | |
|---|---|---|
| | B1 | F1 |
| Disodium ethylenediaminetetraacetate dihydrate | 0.0088 | 0.0084 |
| Cobalt (II) chloride hexahydrate | 0.0026 | 0.0025 |
| Manganese (II) chloride tetrahydrate | 0.0157 | 0.015 |
| Copper (II) chloride dihydrate | 0.00157 | 0.0015 |
| Boric acid | 0.0031 | 0.003 |
| Sodium molybdate dihydrate | 0.0026 | 0.0025 |
| Zinc acetate dihydrate | 0.0136 | 0.0136 |
| Potassium dihydrogen phosphate | 1.0484 | 1.000 |
| Ammonium sulfate | 0.5242 | 0.5 |
| Magnesium sulfate heptahydrate | 0.5242 | 0.5 |
| Calcium chloride dihydrate | 0.0210 | 0.02 |
| Ferrous sulfate heptahydrate | 0.0105 | 0.01 |
| Thiamine hydrochloride | 0.0121 | 0.0115 |
| Sodium nitrate | 0.6290 | 0.6 |
| Glucose | 50 | 100 |

In these cultures, the alanine yield (Yₐₗₐₙᵢₙₑ) was expressed as followed:

$$Y a l a n i n e \left(\frac{g}{g}\right) = \frac{a l a n i n e p r o d u c e d \left(g\right)}{g l u c o s e c o n s u m e d \left(g\right)} * 100$$

Amino acid quantification conditions.

Extracellular amino acids were quantified by HPLC after o-phthalaldehyde/fluorenylmethyl-chloroformate (OPA/FMOC) derivatization and other relevant metabolites were analyzed using HPLC with refractometric detection (organic acids and glucose) and GC-MS after silylation.

### Example 1: Strain Construction

To prepare the strain for alanine production, combinations of mutations were introduced into the E. coli strain resulting in strain 1: MG1655 DackA+pta DadhE DldhA DfrdABCD DmgsA DpflAB, constructed as follows.

To inactivate the ackA+pta, frdABCD, pflAB operons and the adhE, ldhA and mgsA genes, the homologous recombination strategy was used (according to Protocol 1). The strains retained were designated MG1655 DackA+pta::Gt, MG1655 DadhE::Cm, MG1655 DldhA::Km, MG1655 DfrdABCD::Gt, MG1655 DmgsA::Km and MG1655 DpflAB::Cm, where Km, Cm, and Gt designate respectively DNA sequences conferring resistance to kanamycin, chloramphenicol, and gentamycin. All of these deletions were transferred by P1 phage transduction (according to Protocol 2) into E. coli MG1655 and resistance genes were removed according to protocol 3 when necessary, giving rise to strain 1.

To produce alanine, the alaD gene from Geobacillus stearothermophilus (SEQ ID NO: 20) was cloned under the artificial trc promoter without the operator sequence for IPTG induction, into the pME101VB06 plasmid described in patent application EP 2532751 (see in particular Examples 3-6, incorporated herein by reference). The resulting plasmid, pNX0001, was then transformed into the strain 1 giving rise to strain 2.

To overexpress the alanine exporter, the alaE gene from E. coli was cloned with its native promoter into the pNX0001 plasmid. The resulting plasmid, pAL0006, was then transformed into strain 2 giving rise to strain 7.

To overexpress the amino acid exporter, the yddG gene from E. coli was cloned with its native promoter into the pACYC184 plasmid (Chang and Cohen, 1978). The resulting plasmid, pAL0009, was then transformed into strain 2 giving rise to strain 12 and into strain 7 giving rise to strain 17.

The native lrp gene was replaced by diverse mutated lrp alleles using the homologous recombination strategy (according to Protocol 1) giving rise to strains MG1655 Irp*(L74F)::Cm, MG1655 Irp*(L108F)::Cm, MG1655 Irp*(F113C)::Cm, and MG1655 Irp*(123PD)::Cm (introduction of proline and aspartic acid amino acids after position 123). All of these mutated lrp alleles were transferred by P1 phage transduction (according to Protocol 2) into the defined strain and resistance genes were removed according to protocol 3. When necessary, the plasmids were transformed into previous strains giving rise to the new strains described below (Table 2).

TABLE 2

Strains obtained

| Lrp* allele | into strain 2 | into strain 7 | Strains 3 to 6 with pAL0009 | Strains 8 to 11 with pAL0009 |
|---|---|---|---|---|
| L74F | 3 | 8 | 13 | 18 |
| L108F | 4 | 9 | 14 | 19 |
| F113C | 5 | 10 | 15 | 20 |
| 123PD | 6 | 11 | 16 | 21 |

### EXAMPLE 2: Strain Performances With the Diverse Mutated Lrp Alleles

Production strains were assessed in culture conditions as previously described.

TABLE 3

Alanine titer, productivity, and yield for the different strains with the mutated lrp.

| | | | |
|---|---|---|---|
| Strain no. | Titer (g/L) | Prod (g/L/h) | Yield (g/g) |
| Strain 3 | ++ | +++ | + |
| Strain 4 | ++ | +++ | + |
| Strain 5 | ++ | +++ | + |
| Strain 6 | ++ | +++ | + |

The symbol “+” indicates an increase of a factor up to 2, the symbol “++” an increase by a factor between 2 and 5, and “+++” an increase by a factor greater than 5, as compared to the values of reference strain 2.

As can be seen in Table 3, strains 3 to 6 showed an increase in titer, productivity, and yield for alanine as compared to strain 2. Performances were increased with the replacement of the native Irp by diverse mutated lrp alleles. The effect was equivalent with all of the mutated lrp alleles tested.

### EXAMPLE 3: Strain Performances With Overexpression of the Amino Acid Exporter (yddG) in the Mutated Lrp Alleles

Production strains were assessed in culture conditions as previously described.

TABLE 4

Alanine titer, productivity, and yield, for the different strains overexpressing the amino exporter (yddG).

| | | | |
|---|---|---|---|
| Strain no. | Titer (g/L) | Prod (g/L/h) | Yield (g/g) |
| Strain 12 | + | ++ | + |
| Strain 13 | ++ | +++ | + |
| Strain 14 | ++ | +++ | + |
| Strain 15 | ++ | +++ | + |
| Strain 16 | ++ | +++ | + |

The symbol “+” indicates an increase of a factor up to 2, the symbol “++” an increase by a factor between 2 and 5, and “+++” an increase by a factor greater than 5, as compared to the values of reference strain 2.

As can be seen in Table 4, overexpression of the amino acid exporter yddG in strain 2 (strain 12) improves the performance of the strain. Additionally, with the replacement of the native Irp by diverse mutated lrp alleles the performance of strains 13 to 16 was further increased. The effect was equivalent with all of the mutated Irp alleles tested.

### EXAMPLE 4: Strain Performances With Overexpression of the Alanine Exporter (alaE) With and Without Mutated Lrp Alleles

Production strains were assessed in culture conditions as previously described.

TABLE 5

Alanine titer, productivity, and yield for the different strains overexpressing the alanine exporter (alaE).

| | | | |
|---|---|---|---|
| Strain no. | Titer (g/L) | Prod (g/L/h) | Yield (g/g) |
| Strain 7 | + | ++ | + |
| Strain 8 | ++ | +++ | + |
| Strain 9 | ++ | +++ | + |
| Strain 10 | ++ | +++ | + |
| Strain 11 | ++ | +++ | + |

The symbol “+” indicates an increase of a factor up to 2, the symbol “++” an increase by a factor between 2 and 5, and “+++” an increase by a factor greater than 5, as compared to the values of reference strain 2.

As can be seen in Table 5, overexpression of the alanine exporter in strain 2 (strain 7) improves the performance of the strain. Additionally, with the replacement of the native lrp by diverse mutated lrp alleles the performance of strains 8 to 11 was further increased. The effect was equivalent with all of the mutated lrp alleles tested.

### EXAMPLE 5: Strain Performances With Overexpression of the Alanine Exporter (alaE) and the Amino Acid Exporter (yddG) With and Without the Mutated Lrp Alleles

Production strains were assessed in culture conditions as previously described.

TABLE 6

Alanine titer, productivity, and yield, for the different strains overexpressing the alanine exporter (alaE) and the amino acid exporter (yddG).

| | | | |
|---|---|---|---|
| Strain no. | Titer (g/L) | Prod (g/L/h) | Yield (g/g) |
| Strain 17 | ++ | ++ | + |
| Strain 18 | ++ | +++ | + |
| Strain 19 | ++ | +++ | + |
| Strain 20 | ++ | +++ | + |
| Strain 21 | ++ | +++ | + |

The symbol “+” indicates an increase of a factor up to 2, the symbol “++” an increase by a factor between 2 and 5, and “+++” an increase by a factor greater than 5, as compared to the values of reference strain 2.

As can be seen in Table 6, overexpression of the amino acid exporter and the alanine exporter in strain 2 (strain 17) improves the performance of the strain. Additionally, with the replacement of the native Irp by diverse mutated lrp alleles the performances of strains 18 to 21 were further increased. The effect was equivalent with all of the mutated lrp alleles tested.

### REFERENCES

Anderson, (1946), Proc. Natl. Acad. Sci. USA, 32:120-128.
Bantscheff et al., (2007), Anal Bioanal Chem. 389(4):1017-31.
Burnette, (1981), Anal Biochem. 112(2):195-203.
Chang and Cohen, (1978), J Bacteriol. 134(3):1141-56.
Datsenko KA & Wanner BL, (2000), Proc Natl Acad Sci USA., 97: 6640-6645.
Davis and Olsen, (2011), Mol. Biol. Evol.; 28(1):211-221.
Deml, et al., (2001), J. Virol., 75(22): 10991-11001.
Engvall and Perlman, (1971), Immunochemistry; 8(9):871-4.
Gmelch, et al., (2019), Sci Rep. 9, 11754.
Graf et al., (2000), J. Virol.; 74(22): 10/22-10826.
Ihara et al., (2017), Journal of Bioscience and Bioengineering. 123(4): 444-450
Leuchtenberger et al., (2005), Appl Microbiol Biotechnol., 69: 1-8.
Miller, (1992), “A Short Course in Bacterial Genetics: A Laboratory Manual and Handbook for Escherichia coli and Related Bacteria”, Cold Spring Harbor Laboratory Press, Cold Spring Harbor, New York.
Needleman and Wunsch, (1972), J. Mol. Biol., 48(3), 443-453.
Pleissner et al., (2011), Anal. Chem., 83(1) : 175-181.
Prescott et al., (1999), “Microbiology” 4th Edition, WCB McGraw-Hill.
Sambrook and Russell, (2001), Molecular Cloning: 3rd edition, Cold Spring Harbor Laboratory Press, NY, Vol 1, 2, 3.
Sato et al., (1982) Production of L-Alanine from Ammonium Fumarate Using Two Types of Immobilized Microbial Cells. In: Chibata I., Fukui S., Wingard L.B. (eds) Enzyme Engineering. Springer, Boston, MA.
Schaefer et al., (1999), Anal. Biochem., 270: 88-96.
Segel (1993), Enzyme kinetics, John Wiley & Sons, pp. 44-54 and 100-112.
The Expresswire. (2020, June 10). L-Alanine Market SizeAiming on Current Market Conditions, Competitors, Product Price, Profit, Capacity, Production and Future Forecast to 2026 [Press release]. Retrieved from https://www.theexpresswire.com/pressrelease/2020-L-Alanine-Market-Size-Aiming-on-Current-Market-Conditions-Competitors-Product-Price-Profit-Capacity-Production-and-Future-Forecast-to-2026_11161692
Wendisch (2014), Curr. Opin. Biotechnol. 30,51-58.
Zhang et al., (2007), Appl Microbiol Biotechnol. 77(2):355-366.

1. Microorganism genetically modified for the production of alanine, wherein the microorganism expresses a heterologous alaD gene coding an alanine dehydrogenase and has reduced Lrp transcription factor activity and/or expression as compared to the transcription factor activity and/or expression level in a corresponding wild-type microorganism.

2. Microorganism of claim 1, wherein the microorganism comprises an lrp gene coding for an Lrp* mutant having reduced transcription factor activity as compared to the transcription factor activity in a corresponding wild-type microorganism.

3. Microorganism of claim 2, wherein the Lrp* mutant comprises at least one mutation selected from the group consisting of L108F, L74F, F113C, and 123PD, wherein the positions of the amino acid residues correspond to those provided in SEQ ID NO: 1, and wherein mutation 123PD corresponds to the introduction of proline and aspartic acid amino acids after the amino acid residue at position 123.

4. Microorganism of claim 1, comprising at least a partial deletion of the lrp gene, preferably a complete deletion of the lrp gene.

5. Microorganism of claim 1, wherein the microorganism further comprises an overexpression of the yddG gene as compared to the expression level in a corresponding wild-type microorganism.

6. Microorganism of claim 1, further comprising expression of an alaE gene coding an L-alanine exporter at a level similar to that of the corresponding microorganism which does not comprise the genetic modifications according to claim 1 .

7. Microorganism of claim 1, wherein the microorganism expresses the alaD gene of Geobacillus stearothermophilus, Klebsiella aerogenes or Archaeoglobus fulgidus.

8. Microorganism of claim 7, wherein the alaD gene codes the alanine dehydrogenase of SEQ ID NO: 15, 17, 19, 23, or 27.

9. Microorganism of claim 1, further comprising a deletion of at least one gene selected from the group consisting of ackA-pta, ldhA, adhE, frdABCD, mgsA, and pflAB.

10. Microorganism of claim 9, wherein the microorganism comprises the deletion of genes ackA-pta, ldhA, adhE, frdABCD, mgsA, and pflAB.

11. Microorganism of claim 10, further comprising a deletion of the cycA and/or dadX gene(s).

12. Microorganism of claim 1, wherein said microorganism belongs to the family of bacteria Enterobacteriaceae, Clostridiaceae, Bacillaceae, Streptomycetaceae, or Corynebacteriaceae, or to the family of yeasts Saccharomycetaceae.

13. Microorganism of claim 12, wherein said Enterobacteriaceae bacterium is Escherichia coli or Klebsiella pneumoniae, said Clostridiaceae bacterium is Clostridium acetobutylicum, said Corynebacteriaceae bacterium is Corynebacterium glutamicum, or said Saccharomycetaceae yeast is Saccharomyces cerevisiae.

14. Method for the production of alanine comprising the steps of:
a) culturing a microorganism genetically modified for the production of alanine according to claim 1 in an appropriate culture medium comprising a source of carbon, and
b) recovering alanine from the culture medium.

15. Method of claim 14, wherein the source of carbon is selected from arabinose, fructose, galactose, glucose, lactose, maltose, sucrose, xylose, and any combination thereof.

16. Method of claim 14, wherein the microorganism genetically modified for the production of alanine further comprises an overexpression of the yddG gene as compared to the expression level in a corresponding wild-type microorganism.

17. Method of claim 14, wherein the microorganism genetically modified for the production of alanine further comprising an alaE gene coding an L-alanine exporter at a level similar to that of the corresponding microorganism which does not comprise a genetic modification for the expression of a heterologous alaD gene coding an alanine dehydrogenase and having reduced Lrp transcription factor activity and/or expression as compared to the transcription factor activity and/or expression level in a corresponding wild-type microorganism.

18. Microorganism of claim 6, wherein the expression of an alaE gene coding an L-alanine exporter at a level similar to that of the corresponding microorganism is by modifying the alaE promoter or by increasing the number of copies of the alaE gene present in the microorganism.

19. Microorganism of claim 13, wherein said Enterobacteriaceae bacterium is Escherichia coli.

20. Method of claim 17, wherein the alaE gene coding an L-alanine exporter at a level similar to that of the corresponding microorganism which does not comprise a genetic modification for the expression of a heterologous alaD gene coding an alanine dehydrogenase and having reduced Lrp transcription factor activity and/or expression as compared to the transcription factor activity and/or expression level in a corresponding wild-type microorganism, is by modification of the alaE promoter or by increasing the number of copies of the alaE gene present in the microorganism.


SEQUENCE LISTING (47 sequences)

SEQ ID NO: 1 - protein, 164 aa, Escherichia coli
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHLVSGDFDYLLKTRVPDMSAYRKLLGETLLRLPGVNDTRTYVVMEEVKQSNRLVIKTR

SEQ ID NO: 2 - DNA, 495 bp, Escherichia coli
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatccggtgatttcgactacctgttgaaaacacgcgtgccggatatgtcagcctaccgtaagttgctgggggaaaccctgctgcgtctgcctggcgtcaatgacacacggacatacgtcgttatggaagaagtcaagcagagtaatcgtctggttattaagacgcgctaa

SEQ ID NO: 3 - protein, 164 aa, Artificial Sequence
  Lrp* (L108F)
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHFVSGDFDYLLKTRVPDMSAYRKLLGETLLRLPGVNDTRTYVVMEEVKQSNRLVIKTR

SEQ ID NO: 4 - DNA, 495 bp, Artificial Sequence
  Lrp* (L108F)
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcattttgtatccggtgatttcgactacctgttgaaaacacgcgtgccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 5 - protein, 164 aa, Artificial Sequence
  Lrp* (L74F)
MVDSKKRPGKDLDRIDRNILNELQKDGRISNVELSKRVGLSPTPCLERVRRLERQGFIQGYTALLNPHYLDASFLVFVEITLNRGAPDVFEQFNTAVQKLEEIQECHLVSGDFDYLLKTRVPDMSAYRKLLGETLLRLPGVNDTRTYVVMEEVKQSNRLVIKTR

SEQ ID NO: 6 - DNA, 495 bp, Artificial Sequence
  Lrp* (L74F)
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcatttctggtattcgttgagattactctgaatcgtggcgcaccggatgtgtttgaacaattcaataccgctgtacaaaaacttgaagaaattcaggagtgtcatttagtatccggtgatttcgactacctgttgaaaacacgcgtgccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 7 - protein, 164 aa, Artificial Sequence
  Lrp* (F113C)
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHLVSGDCDYLLKTRVPDMSAYRKLLGETLLRLPGVNDTRTYVVMEEVKQSNRLVIKTR

SEQ ID NO: 8 - DNA, 495 bp, Artificial Sequence
  Lrp* (F113C)
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatccggtgattgcgactacctgttgaaaacacgcgtgccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 9 - protein, 166 aa, Artificial Sequence
  Lrp* (123PD)
MVDSKKRPGKDLDRIDRNILNELQKDGRISNVELSKRVGLSPTPCLERVRRLERQGFIQGYTALLNPHYLDASLLVFVEITLNRGAPDVFEQFNTAVQKLEEIQECHLVSGDFDYLLKTRVPDPDMSAYRKLLGETLLRLPGVNDTRTYVVMEEVKQSNRLVIKTR

SEQ ID NO: 10 - DNA, 501 bp, Artificial Sequence
  Lrp* (123PD)
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatccggtgatttcgactacctgttgaaaacacgcgtgccggatccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 11 - protein, 369 aa, Thermus thermophilus
MVIGVPKEIKTLENRVALTPGGVESLVRRGHTVLVERGAGEGSGLSDAEYARAGAELVGREEAWGAEMVVKVKEPLPEEYGFLREGLILFTYLHLAADRGLTEAMLRSGVTGIAYETVQLPDGTLPLLVPMSEVAGRMAPQVGAQFLEKPKGGRGVLLGGVPGVAPASVVILGGGTVGTNAAKIALGMGAQVTILDVNHKRLQYLDDVFGGRVITLTATEANIKKSVQHADLLIGAVLVPGAKAPKLVTRDMLSLMKEGAVIVDVAVDQGGCVETIRPTTHAEPTYVVDGVVHYGVANMPGAVPRTSTFALTNQTLPYVLKLAEKGLDALLEDAALLKGLNTHKGRLTHPGVAEAFGLPYTPPEEALRG

SEQ ID NO: 12 - DNA, 1110 bp, Thermus thermophilus
atggtgatcggcgtgccgaaggagatcaagaccttggagaaccgggtggccctcacgcccggcggggtggagagcctggtcaggcgcggccacaccgtgctggtggagcggggggccggggagggctcggggctttccgacgcggagtacgcccgggccggggccgagctcgtgggccgggaggaggcctggggtgcggagatggtggtgaaggtgaaggagcccctacccgaggagtacggcttcctgcgggagggcctcatcctcttcacctaccttcacctggccgcggaccgcggcctcaccgaggccatgctccgtagcggggtcacgggcatcgcctacgagaccgtccagcttcccgacggcaccctccccctcctcgtccccatgagcgaggtggcggggcggatggccccccaggtgggggcccagttcctggagaagcccaaggggggccggggggtcctcctcgggggggtgccgggggtggccccggccagcgtggtgatcctcgggggcgggaccgtgggcaccaacgcggccaagatcgccctggggatgggggcccaggtgaccatcctggacgtgaaccacaagcgcctccagtatctggacgacgtcttcggcgggcgggtgatcaccctcaccgccaccgaggccaacatcaaaaagagcgtccagcacgcggacctcctcatcggggccgtcctcgtccccggggccaaggcccccaagctcgtcacccgggacatgctctctctgatgaaggagggagcggtgatcgtggacgtggccgtggaccagggggggtgcgtggagaccatccggcccaccacccacgccgagcccacctacgtggtggacggggtggtccactacggggtggccaacatgcccggggcggtgcccaggaccagcaccttcgccctcaccaaccagaccctgccctacgtgttgaagctcgcggagaaggggctggacgcccttctggaggacgcggcccttctcaaggggctcaacacccacaaaggccgcctcacccaccccggggtggccgaggccttcggcctgccctacacgcctcccgaggaggccttgagggggtga

SEQ ID NO: 13 - protein, 372 aa, Lysinibacillus sphaericus
MKIGIPKEIKNNENRVAMTPAGVVSLTHAGHERLAIETGGGIGSSFTDAEYVAAGAAYRCIGKEAWAQEMILKVKEPVASEYDYFYEGQILFTYLHLAPRAELTQALIDKKVVGIAYETVQLANGSLPLLTPMSEVAGKMATQIGAQYLEKNHGGKGILLGGVSGVHARKVTVIGGGIAGTNAAKIAVGMGADVTVIDLSPERLRQLEDMFGRDVQTLMSNPYNIAESVKHSDLVVGAVLIPGAKAPKLVSEEMIQSMQPGSVVVDIAIDQGGIFATSDRVTTHDDPTYVKHGVVHYAVANMPGAVPRTSTIALTNNTIPYALQIANKGYKQACIDNPALKKGVNALEGHITYKAVAEAQGLPYVNVDELIQ

SEQ ID NO: 14 - DNA, 1119 bp, Lysinibacillus sphaericus
atgaagattggtattccaaaggaaattaaaaacaacgaaaatcgcgtagcaatgacaccagcaggagttgtatccttaacgcatgctgggcacgagaggttagctattgaaacaggaggtggtatcggttcaagttttacagatgcagagtacgtagcagcaggtgcagcatatcgttgcatcggcaaagaagcgtgggctcaagaaatgattttaaaagtaaaagaacccgtagcatcggaatacgattacttttatgagggacaaatcttatttacctacttgcacttagcgcccagagctgaattaacgcaggcattaatagataaaaaagttgtaggtattgcctatgaaacggttcaacttgcaaatggttcactacctttattaacaccaatgagtgaagtagctggtaaaatggcaacacaaattggtgcgcaatatttagagaaaaatcacggtggtaaagggattttactaggcggtgtatcaggtgtacacgcgcgtaaagtaacagtaattggtggcggaatcgcgggaacaaacgctgcgaaaattgcagttggtatgggagcagacgtaacagttattgatttaagtccagaacgtctacgtcaattagaagatatgtttggtcgcgatgttcaaacattaatgtctaacccgtataatattgcagaatctgtgaaacactcagatttagttgtcggtgctgttctaattcctggtgcaaaggctccaaagttagtttcggaagaaatgattcaatcgatgcaaccaggttctgttgttgtggatattgcgattgaccaaggtggaatttttgcgacatctgatcgtgttacaacacatgatgatccaacgtatgttaaacatggggtagtccattatgctgttgcaaatatgccaggggctgtgccacgtacttcaacgattgctttaacaaataatacaattccttatgcgttgcaaattgccaataaaggctataagcaagcatgtattgacaatcctgcattgaaaaaaggtgtgaatgcattagaggggcatattacttataaagcggtagcagaagcacaaggcttgccatatgtgaatgtggatgaattaatccaataa

SEQ ID NO: 15 - protein, 372 aa, Geobacillus stearothermophilus
MKIGIPKEIKNNENRVAITPAGVMTLVKAGHEVYVETEGGAGSGFSDSEYEKAGAADRCRTWRDAWTAEMVLKVKEPLAREFRYFRPGLILFTYLHLAAAERVTKAVVEQKVVGIAYETVQLANGSLPLLTPMSEVAGRMSVQVGAQFLEKPHGGKGILLGGVPGVRRGKVTIIGGGTAGTNAAKIGVGLGADVTILDINAERLRELDDLFGDHVTTLMSNSYHIAECVRESDLVVGAVLIPGAKAKLVTEEMVRSMTPGSVLVDIAIDQGGIFETTDRVTTHDDPTYVKHGVVHYAVANMPGAVPRTSTFALTNVTIPYALQIANKGYRAGCLDNPALLKGINTLDGHIVYEAVAAAHNMPYTDVHSLLHG

SEQ ID NO: 16 - DNA, 1119 bp, Geobacillus stearothermophilus
atgaagatcggcattccaaaagaaatcaaaaacaatgaaaaccgcgtcgccatcactccggcaggcgtgatgacgctcgtcaaagcggggcatgaggtgtatgtggagacggaaggcggcgctgggtcggggttttccgattccgagtatgaaaaagccggggcagctgatcgttgccgaacgtggagagatgcttggacggcggagatggtgttgaaagtgaaagagccgctggctcgagagttccgctattttcgccccggattgattttgtttacgtatttgcatttagccgcggccgaacgcgtcacgaaagccgtcgtcgagcaaaaagtggtcggcatcgcttacgagacggtgcagctggcgaacggctcgctgccactgttgacgccgatgagtgaagtcgccggccgcatgtcggtgcaagtcggcgcccagtttctcgagaagccgcacgggggaaagggcattttgcttggcggcgtgcccggagtgcggcgcggcaaagtgacgatcatcggcggcggaacggcggggacgaacgcggcgaaaatcggggtcggtctcggtgcagacgtgacgattttggacattaacgccgagcggctgcgcgagctcgatgatttgttcggcgaccacgtgacgacgctcatgtccaactcgtaccatatcgccgagtgcgtgcgcgaatcggatttggtcgtcggtgccgtcttgatcccgggggcgaaagcgaagctggtgacggaagagatggtgcgctcgatgacgccgggatcggtgttggtcgacatcgccattgaccaaggcggcattttcgaaacgaccgaccgcgtcacgacgcacgacgatccgacatacgtcaagcacggcgtcgtccattacgccgtcgccaacatgccgggcgcggtgccgcgcacgtcgacattcgcgcttacgaacgtcacgatcccatacgccttgcaaatcgccaacaaaggctaccgcgccggttgcttggataacccggcgctgttaaaagggatcaacacgctcgacgggcacatcgtgtacgaagcggtcgcggcggcgcacaacatgccgtatacagatgttcattcgttgttgcacggatga

SEQ ID NO: 17 - protein, 372 aa, Geobacillus stearothermophilus
MKIGIPKEIKNNENRVAITPAGVMTLVKAGHDVYVETEAGAGSGFSDSEYEKAGAVIVTKAEDAWAAEMVLKVKEPLAEEFRYFRPGLILFTYLHLAAAEALTKALVEQKVVGIAYETVQLANGSLPLLTPMSEVAGRMSVQVGAQFLEKPHGGKGILLGGVPGVRRGKVTIIGGGTAGTNAAKIAVGLGADVTILDINAERLRELDDLFGDQVTTLMSNSYHIAECVRESDLVVGAVLIPGAKAPKLVTEEMVRSMTPGSVLVDVAIDQGGIFETTDRVTTHDDPTYVKHGVVHYAVANMPGAVPRTSTFALTNVTIPYALQIANKGYRAACLDNPALLKGINTLDGHIVYEAVAAAHNMPYTDVHSLLQG

SEQ ID NO: 18 - DNA, 1119 bp, Geobacillus stearothermophilus
atgaagatcggcattccaaaagaaatcaaaaacaatgaaaaccgcgtcgccatcactccggcaggcgtgatgacgctcgtcaaagcggggcatgacgtgtatgtggagacggaagccggcgctgggtcgggtttttccgattccgagtatgaaaaagccggggcagtgatcgtgacgaaagcggaagatgcctgggcggcggagatggtgttgaaagtgaaagaaccgctggctgaggagttccgctattttcgccccggattgattttgtttacgtatttgcatttagccgcggccgaagcgctcacgaaagcgctcgtcgagcaaaaagtggtcggcatcgcttacgagacggtgcagcttgcgaacggctcgctgccgctgttgacgccgatgagtgaagtcgccggccgcatgtcggtgcaagtcggcgcccagtttctcgagaagccgcacggcgggaaaggcattttgcttggcggcgtgcccggggtgcggcgcggcaaagtgacgatcatcggcggcggcacagcggggacgaacgcggcgaaaatcgcggtcggcctcggggcggacgtgacgattttggacattaacgccgagcggctgcgcgagctcgatgatttgttcggcgaccaagtgacgacgttgatgtccaactcgtatcatatcgccgagtgcgtgcgcgaatccgatttggtcgtcggcgccgtcttgatcccgggggcgaaagcgccgaagcttgtgacggaagagatggtgcgctcgatgacgccaggctcggtgttggtcgacgtcgccattgaccaaggcggcatttttgaaacgaccgaccgcgtcacgacgcacgacgatccgacatacgtcaagcacggcgtcgtccattacgccgtcgcgaacatgccgggcgctgtgccgcgtacgtcaacattcgcgcttacgaacgtcacgatcccatacgccttgcaaatcgccaacaaaggctaccgcgccgcttgcctcgacaatccggcgctgttaaaagggatcaacacgctcgacgggcacatcgtgtacgaagcggtcgcggcggcgcacaacatgccgtatacggatgttcattcgttgctgcagggatga

SEQ ID NO: 19 - protein, 372 aa, Geobacillus stearothermophilus
MKIGIPKEIKNNENRVAITPAGVMTLVKAGHDVYVETEAGAGSGFSDSEYEKAGAVIVPNAEDAWTAEMVLKVKEPLAEEFRYFRPGLILFTYLHLAAAEALTKALVEQKVVGIAYETVQLANGSLPLLTPMSEVAGRMSVQVGAQFLEKPHGGKGILLGGVPGVRRGKVTIIGGGTAGTNAAKIAVGLGSDVTILDINAERLRELDDLFGDHVTTLMSNSYHTAECVRESDLVVGAVLIPGAKAPKLVTEEMVRSMTPGSVLVDIAIDQGGIFETTDRVTTHDDPTYVKHGVVHYAVANMPGAVPRTSTFALTNVTIPYALQIANKGYRAACLDNPALLKGINTLDGHIVYEAVAAAHNMPYTDVHSLLHG

SEQ ID NO: 20 - DNA, 1119 bp, Geobacillus stearothermophilus
atgaagatcggcattccaaaagaaatcaaaaacaatgaaaaccgcgtcgccatcactccggcaggcgtgatgacgctcgtcaaagcggggcatgacgtgtatgtggagacggaagccggcgctgggtcggggttttccgattccgagtatgaaaaagccggggcagtgatcgtgccgaacgcggaagatgcttggacggcggagatggtgttgaaagtgaaagagccgctggctgaggagttccgctattttcgccccggattgattttgtttacgtatttgcatttagccgcggccgaagcgctcacgaaagcgctcgtcgagcaaaaagtggtcggcatcgcttacgagacggtgcagctggcgaacggctcgctaccactgttgacgccgatgagtgaagtcgccggccgcatgtcggtgcaagtcggcgcccagtttctcgagaagccgcacggcgggaaaggcattttgcttggcggcgtgcccggagtgcggcgcggcaaagtgacgatcatcggcggcggaacggcggggacgaacgcggcgaaaatcgcggtcggtctcgggtcagacgtgacgattttggacattaacgccgagcggctgcgcgagctcgatgatttgttcggcgaccacgtgacgacgctcatgtccaactcgtaccataccgccgagtgcgtgcgcgaatcggatttggtcgtcggtgccgtcttgatcccgggggcgaaagcgccgaagctggtgacggaagagatggtgcgctcgatgacgccgggatcggtgttggtcgacatcgccattgaccaaggcggcattttcgaaacgaccgaccgcgtcacgacgcacgacgatccgacatacgtcaagcacggcgtcgtccattacgccgtcgccaacatgccgggcgcggtgccgcgcacgtcgacattcgcgcttacgaacgtcacgatcccatacgccttgcaaatcgccaacaaaggctaccgcgccgcgtgcttggataacccggcgctgttaaaggggatcaacacgctcgacgggcacatcgtgtacgaagcggtcgcggcggcgcacaacatgccgtatacagatgttcattcgttgttgcacggatga

SEQ ID NO: 21 - protein, 371 aa, Mycobacterium tuberculosis
MRVGIPTETKNNEFRVAITPAGVAELTRRGHEVLIQAGAGEGSAITDADFKAAGAQLVGTADQVWADADLLLKVKEPIAAEYGRLRHGQILFTFLHLAASRACTDALLDSGTTSIAYETVQTADGALPLLAPMSEVAGRLAAQVGAYHLMRTQGGRGVLMGGVPGVEPADVVVIGAGTAGYNAARIANGMGATVTVLDINIDKLRQLDAEFCGRIHTRYSSAYELEGAVKRADLVIGAVLVPGAKAPKLVSNSLVAHMKPGAVLVDIAIDQGGCFEGSRPTTYDHPTFAVHDTLFYCVANMPASVPKTSTYALTNATMPYVLELADHGWRAACRSNPALAKGLSTHEGALLSERVATDLGVPFTEPASVLA

SEQ ID NO: 22 - DNA, 1122 bp, Mycobacterium tuberculosis
atgcgcgtcggtattccgaccgagaccaaaaacaacgaattccaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctga

SEQ ID NO: 23 - protein, 322 aa, Archaeoglobus fulgidus
METLILTQEEVESLISMDEAMNAVEEAFRLYALGKAQMPPKVYLEFEKGDLRAMPAHLMGYAGLKWVNSHPGNPDKGLPTVMALMILNSPETGFPLAVMDATYTTSLRTGAAGGIAAKYLARKNSSVFGFIGCGTQAYFQLEALRRVFDIGEVKAYDVREKAAKKFVSYCEDRGISASVQPAEEASRCDVLVTTTPSRKPVVKAEWVEEGTHINAIGADGPGKQELDVEILKKAKIVVDDLEQAKHGGEINVAVSKGVIGVEDVHATIGEVIAGLKDGRESDEEITIFDSTGLAIQDVAVAKVVYENALSKNVGSKIKFFRI

SEQ ID NO: 24 - DNA, 969 bp, Archaeoglobus fulgidus
atggagactcttattttgactcaggaggaagtagaatccctgatatcgatggacgaggcgatgaatgctgttgaggaggcgttcagactttacgctcttggtaaagctcaaatgccgccaaaggtgtatcttgagttcgaaaaaggcgatttaagggctatgccggcgcatttaatggggtacgcaggattgaagtgggtgaactcccatcccggaaatccggataaaggcctgccaacagtcatggcgttgatgatactgaacagccctgagacggggtttcccttggctgtgatggacgcaacctacacgacctctctgagaaccggtgctgctggaggaatagcggcgaagtatctggcaaggaagaacagcagcgtttttggatttatcggctgcggaactcaggcgtatttccagctcgaagctctgaggagagtgtttgatataggagaagtcaaggcctacgatgttagggaaaaggcagcgaaaaagttcgttagctattgcgaagataggggaatctcagcttccgtgcagcctgctgaggaggcgagcagatgcgatgtccttgtcaccacaactccgtcacgaaaaccagttgttaaggctgagtgggttgaggaggggacgcacataaacgcaatcggtgcggacgggcctggaaagcaggaactggatgtagaaatacttaaaaaggctaaaattgttgtcgatgaccttgaacaggccaagcacggcggggagattaatgtggcggtttcaaaaggcgtcataggtgttgaagatgttcacgcaaccatcggggaggttatagccggtctgaaggatggaagggagagcgatgaggagattacaatcttcgactccacaggcttggcgattcaggacgtggctgttgcaaaagtcgtttatgagaacgcccttagcaagaacgtggggagtaaaataaagtttttcaggatatga

SEQ ID NO: 25 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 26 - DNA, 1137 bp, Bacillus subtilis
atgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgacttaaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

SEQ ID NO: 27 - protein, 377 aa, Klebsiella aerogenes
MIIGVPKEIKNNENRVAMTPAGVVHLLNAGHKVIIETNAGLGSGFTNEEYKQAGAEIIESASDVWTKADMIMKVKEPLASEYGYFRKGLILFTYLHLAAEPELTKALVDSEVIAIAYETVTVNRTLPLLSPMSEVAGRMAAQVGAQFLEKTQGGKGILLSGVPGVKRGKVTIIGGGMVGTNAAKIAVGLGADVTIIDLNPDRLRQLEDIFGTSVQTLMSNPYNIAEAVKESDLVIGSVLIPGAKAPKLVTEEMVKSMQPGSVIVDVAIDQGGNFETVDHITTHDDPTYVKHGVVHYAVANMPGAVPRTATIALTNVTIPYAVQIATKGVVKAVNDNPAIKAGVNVANGHVTFEAVANDLGYKYVTVEEAISKEAINA

SEQ ID NO: 28 - DNA, 1134 bp, Klebsiella aerogenes
atgattatcggtgttcctaaggaaattaaaaataatgaaaatcgtgtggcaatgactcctgctggtgtcgttcatttattaaatgctggacataaagttattattgaaacaaacgctggattaggcagtggttttacgaacgaagaatataaacaagctggtgctgaaattattgaaagcgcttcagatgtatggacgaaagctgatatgattatgaaggttaaagaaccgcttgcatctgagtacggttatttccgtaaaggattaatcttattcacatatcttcacttagctgcagaacctgaattaacaaaagcgttggtagatagtgaagtgattgccattgcatatgaaacagttactgtaaatcgtacattaccacttctttcaccaatgagtgaagtagcaggcagaatggcagcacaagtcggagctcaattccttgaaaagacacaaggtggtaaaggaattctactaagtggtgtacctggagtaaaacgtggcaaagtaacgattattggtggcggaatggttggaacaaacgctgcaaaaatcgcagtcggtctaggagcggatgtaacaatcatcgatttaaatccagatcgtttacgtcaattagaagacatttttggtacaagtgttcaaacattaatgtcaaatccatacaatattgctgaagctgttaaagaatccgatttagttattggatcagtactcattcctggtgcaaaagctccgaaattagtgacagaagaaatggttaaatccatgcaaccaggatctgttattgtagacgttgcaatcgaccaaggtggaaacttcgaaacagttgatcatattacaactcatgatgacccaacttatgttaaacacggtgttgttcattatgcagtagctaatatgcctggtgcagttccacgtacagctacaattgccttaacaaatgtaacgattccttatgctgtacaaattgcgacaaaaggtgtagttaaagcagtgaatgataatcctgcaattaaagctggtgtgaacgtagcgaatggccatgtaacatttgaagcagtagcaaacgatcttggctacaaatatgtaacagtagaagaagcaatttctaaagaagcaatcaatgcataa

SEQ ID NO: 29 - protein, 361 aa, Phormidium lapideum
MEIGVPKEIKNQEFRVGLSPSSVRTLVEAGHTVFIETQAGIGAGFADQDYVQAGAQVVPSAKDAWSREMVVKVKEPLPAEYDLMQKDQLLFTYLHLAAARELTEQLMRVGLTAIAYETVELPNRSLPLLTPMSIIAGRLSVQFGARFLERQQGGRGVLLGGVPGVKPGKVVILGGGVVGTEAAKMAVGLGAQVQIFDINVERLSYLETLFGSRVELLYSNSAEIETAVAEADLLIGAVLVPGRRAPILVPASLVEQMRTGSVIVDVAVDQGGCVETLHPTSHTQPTYEVFGVVHYGVPNMPGAVPWTATQALNNSTLPYVVKLANQGLKALETDDALAKGLNVQAHRLVHPAVQQVFPDLA

SEQ ID NO: 30 - DNA, 1086 bp, Phormidium lapideum
atggaaatcggcgttcccaaagagattaagaatcaggaatttcgggtggggctgagtcccagcagcgtccgcacgctggtcgaagcaggacacacagtatttatcgaaacccaggcgggcatcggcgcaggatttgccgatcaggactatgttcaagctggcgctcaggtcgtgccttctgcaaaggatgcctggagccgagagatggtggtgaaggtcaaggaaccgctgcctgcggaatatgacctgatgcaaaaagatcaattgctgtttacctacctgcacctggcggcggcaagagaactgacggagcaactgatgcgggtggggctgacggcgatcgcctatgaaaccgtcgaactgcccaaccgcagcctgcccctgctgactcccatgagcatcatcgctgggcggctctcggttcagtttggggcccgctttttggaacgacagcagggtgggcgcggcgtgctgctaggcggcgtgccgggggtaaaaccaggcaaagtcgtgatcttgggcggcggtgtggtgggcaccgaagctgccaagatggccgtcgggctgggcgctcaggtgcaaatttttgacatcaatgtggagcggctgtcttacctggaaaccctgtttggctcgcgggtcgagctgctctacagcaattccgccgagatcgagactgcggtcgccgaggctgatttgctcatcggcgcagtgctggtaccgggccgacgtgcgcccatcctagtgcctgccagcctggtggagcaaatgcggacaggctcggtgatcgtagacgtggcggtggatcagggcggctgcgtagagacgctgcacccaacttcccacacgcagcccacctacgaggtgtttggcgtggttcactatggcgtgccgaatatgcccggagccgttccctggacagcgacgcaggcactcaacaacagcaccctgccctacgtggtgaagctggccaaccagggacttaaggcactggaaaccgatgacgcgctagccaaggggctaaacgtgcaggcccatcgcttggtgcatcccgcagtgcagcaggttttccccgatctggcgtag

SEQ ID NO: 31 - protein, 293 aa, Escherichia coli
MTRQKATLIGLIAIVLWSTMVGLIRGVSEGLGPVGGAAAIYSLSGLLLIFTVGFPRIRQIPKGYLLAGSLLFVSYEICLALSLGYAATHHQAIEVGMVNYLWPSLTILFAILFNGQKTNWLIVPGLLLALVGVCWVLGGDNGLHYDEIINNITTSPLSYFLAFIGAFIWAAYCTVTNKYARGFNGITVFVLLTGASLWVYYFLTPQPEMIFSTPVMIKLISAAFTLGFAYAAWNVGILHGNVTIMAVGSYFTPVLSSALAAVLLSAPLSFSFWQGALMVCGGSLLCWLATRRG

SEQ ID NO: 32 - DNA, 882 bp, Escherichia coli
atgacacgacaaaaagcaacgctcatagggctgatagcgatcgtcctgtggagcacgatggtaggattgattcgcggtgtcagtgaggggctcggcccggtcggcggcgcagctgctatctattcattaagcgggctgctgttaatcttcacggttggatttccgcgtattcggcaaatcccgaaaggctatttactcgccgggagtctgttattcgtcagctatgaaatctgtctggcgctttccttagggtatgcggcgacccatcatcaggcgattgaagtgggtatggtgaactatctgtggcccagcctgacaattctctttgccattctgtttaatggtcagaaaaccaactggttgattgtacctggattattattagccctcgtcggcgtctgttgggtgttaggcggtgacaatgggttacattatgatgaaatcatcaataatatcaccaccagcccattgagttatttcctggcgttcattggtgcgtttatctgggcagcctattgcacagtaacgaataaatacgcacgcggatttaatggaattaccgtttttgtcctgctaacgggagcaagtctgtgggtttactattttcttacgccacaaccagaaatgatatttagcacgcccgtcatgattaaactcatctctgcggcatttaccttaggatttgcttatgctgcatggaatgtcggtatattgcatggcaatgtcaccattatggcggtaggttcgtattttacgcctgtactttcctcagcgcttgcagccgtgctgctcagcgccccgctgtcgttctcgttctggcaaggcgcgctgatggtctgcggcggttccctgctctgctggctggcgacacgtcgtggttaa

SEQ ID NO: 33 - protein, 149 aa, Escherichia coli
MFSPQSRLRHAVADTFAMVVYCSVVNMCIEVFLSGMSFEQSFYSRLVAIPVNILIAWPYGMYRDLFMRAARKVSPSGWIKNLADILAYVTFQSPVYVAILLVVGADWHQIMAAVSSNIVVSMLMGAVYGYFLDYCRRLFKVSRYQQVKA

SEQ ID NO: 34 - DNA, 450 bp, Escherichia coli
atgttctcaccgcagtcacgcttgcgtcatgcagttgcagatacgttcgcgatggttgtttactgttctgtcgtgaacatgtgtattgaagttttcctctccggaatgagcttcgaacagtctttttattccagattggtagcgattccggtgaacatcttaattgcatggccatacggtatgtaccgtgatctgtttatgcgcgcggcacgcaaagttagcccgtcgggctggataaaaaatctggcggatatcctggcttatgtgacgttccagtcaccggtgtatgtggcgatcttgttagtggtgggcgcagactggcatcagattatggcggcggtcagttcaaacatcgttgtttcgatgttgatgggggcggtttatggctacttcctcgattattgccgccgactgtttaaagtcagccgttaccagcaggtaaaagcctga

SEQ ID NO: 35 - DNA, 1203 bp, Escherichia coli
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccttacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctgaccaaagagtctggcctgctgggtctgaccgaagtgaccagcgactgccgctatgttgaagacaactacgcgacgaaagaagacgcgaagcgcgcaatggacgtttactgccaccgcctggcgaaatacatcggtgcctacactgcgctgatggatggtcgtctggacgctgttgtattcactggtggtatcggtgaaaatgccgcaatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 36 - DNA, 2145 bp, Escherichia coli
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 37 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 38 - DNA, 1809 bp, Escherichia coli
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 39 - DNA, 735 bp, Escherichia coli
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 40 - DNA, 396 bp, Escherichia coli
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 41 - DNA, 360 bp, Escherichia coli
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

SEQ ID NO: 42 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 43 - DNA, 459 bp, Escherichia coli
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 44 - DNA, 741 bp, Escherichia coli
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 45 - DNA, 2283 bp, Escherichia coli
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 46 - DNA, 1413 bp, Escherichia coli
atggtagatcaggtaaaagtcgttgccgatgatcaggctccggctgaacagtcgctacggcgcaatctcacaaaccgacatattcagcttattgccattggcggtgccattggtacggggttgtttatggggtctggcaaaacgattagccttgccgggccgtcgatcattttcgtttatatgatcattggttttatgctctttttcgtgatgcgggcaatgggggaattgctgctttcgaatctggaatacaaatcttttagtgacttcgcttccgatttactcgggccgtgggcaggatatttcaccggctggacttactggttctgctgggttgtaaccggtatggcagacgtggtggcgatcacggcttatgctcagttctggttccccgatctctccgactgggtcgcctcgctggcggtgatagtgctgctgctgacgctcaatctcgccaccgtgaaaatgttcggtgagatggagttctggtttgcgatgatcaaaatcgtcgccattgtgtcgctgattgtcgtcggcctggtcatggtggcgatgcactttcagtcaccgactggtgtggaagcgtcattcgcgcatttgtggaatgacggcggctggttcccgaaaggtttaagtggcttctttgccggattccagatagcggttttcgctttcgtggggattgagctggtaggtacaacagctgcggaaaccaaagatccagagaaatcactgccacgcgcgattaactccattccgatccgtatcattatgttctacgtcttcgcgctgattgtgattatgtccgtgacgccgtggagttcggtagtcccggagaaaagcccgtttgttgaactgttcgtgttggtagggctgcctgctgccgcaagcgtgatcaactttgtggtgctgacctctgcggcgtcttccgctaacagcggcgtcttctctaccagccgtatgctgtttggtctggcgcaggaaggtgtggcaccgaaagcgttcgctaaactttctaagcgcgcagtacccgcgaaagggctgacgttctcgtgtatctgtctgctcggcggcgtggtgatgttgtatgtgaatcctagtgtgattggcgcgttcacgatgattacaaccgtttccgcgattctgtttatgttcgtctggacgattatcctttgctcgtaccttgtgtatcgcaaacagcgtcctcatctacatgagaagtcgatctacaagatgccgctcggcaagctgatgtgctgggtatgtatggcgttctttgtgttcgtggtcgtgttgctgacactggaagatgacactcgccaggcgctgctggttaccccgctgtggtttatcgcgctggggttgggctggctgtttattggtaagaagcgggctgctgaactgcggaaataa

SEQ ID NO: 47 - DNA, 1071 bp, Escherichia coli
atgacccgtccgatacaggccagcctcgatctgcaggcattaaaacagaatctgtccattgtccgccaggccgcgacgcacgcgcgcgtctggtcggtggtaaaagcgaacgcttacgggcatggtattgagcgtatctggagcgcgatcggggccaccgatggctttgcattgcttaacctggaagaggcaataacgttacgtgagcgcggctggaaaggaccgatcctgatgctggaaggatttttccatgctcaggatctggagatttatgaccagcaccgcctgaccacctgcgtacacagcaactggcagctcaaagcactgcaaaatgcgcggctaaaagcaccgttggatatttatcttaaagtgaacagtgggatgaatcggttgggcttccagcccgatcgcgtgcttaccgtctggcagcagttgcgggcaatggcgaatgttggcgaaatgaccctgatgtcgcattttgccgaagcggaacatcctgatggaatttccggccggatggcgcgtattgagcaggcggcggaggggctggagtgtcggcgttcgttgtccaattcggcggcgactctgtggcacccggaagcgcattttgactgggttcggcctggcattattttgtatggccgttcgccgtccggtcagtggcgtgatatcgccaataccggattacgtccggtgatgacgctaagcagtgagattattggtgtccagacgctaaaagcgggcgagcgtgtgggctacggcggtcgctatactgcgcgcgatgaacagcgaatcggcattgtcgccgcagggtacgccgacggttatccgcgccacgccttaaccggtacccctgttttagtggacggcgtgcgcaccatgacggtggggaccgtctcgatggatatgctagcggtcgatttaacgccttgcccgcaggcgggtattggtacgccggttgagctgtggggcaaggagatcaaaattgatgatgtcgccgccgctgccggaacggtgggctatgagttgatgtgcgcgctggccgtacgcgtcccggttgtgacggtgtaa